(19) United States
(12) Patent Application Publication
(10) Pub. No.: US 2006/0081721 A1
(43) Pub. Date: Apr. 20, 2006

(54) Diffuser of multiple volatile substances

(75) Inventors: Andrea Caserta, Barcelona (ES); Cedric Morhain, Barcelona (ES); David Moreno Perez, Barcelona (ES); Jose Antonio Munoz Martinez, Barcelona (ES)

Correspondence Address: MCDERMOTT WILL & EMERY LLP, 18191 VON KARMAN AVE., SUITE 500, IRVINE, CA, 92612-7108, US

(73) Assignee: ZOBELE ESPANA, S.A., Barcelona (ES)

(21) Appl. No.: 11/073,871

(22) Filed: Mar. 8, 2005

(30) Foreign Application Priority Data
Aug. 13, 2004 (WO) PCT/ES04/00376

(51) Int. Cl. A61L 9/04

(52) U.S. Cl. 239/44; 239/135

(57) ABSTRACT

The invention relates to a diffuser of multiple volatile substances (air freshener liquid, odour absorption formulae, bactericide active substances, insecticides, etc.) comprising two or more containers (2, 2′) placed with a symmetrical or approximately symmetrical arrangement about a central body (1) to which they are joined by corresponding supports (3, 3′) that constitute said central body (1). Each container (2, 2′) holding the air freshener liquid or the desired formulation is closed by an active substance diffuser body (4) that also acts as a stopper and prevents dripping. When the device is at rest only one of the containers is diffusing. To change the fragrance, simply turn the device automatically or manually so that the container that was emanating is in the opposite position with respect to the other container, which will then diffuse its active substance, its evaporation being regulated individually or globally.

### OBJECT OF THE INVENTION

This invention relates to a diffuser of multiple volatile substances or fragrances that comprises at least two containers placed at a relatively symmetrical position with respect to the central body of the diffuser device, with volatile air freshening substances that can have various natures and/or produce different effects, allowing in a simple and fast manner to change the fragrance o fragrances released at a given moment by the diffuser at one of its multiple containers by simply changing the position of the diffuser, turning it by 180° about its vertical axis of symmetry, or by less, so that the diffusion of the substances takes place from another container comprised in the device of the invention.

The invention disclosed can be encompassed in both electrical diffusers of perfumes and other volatile substances and in air fresheners or non-electrical diffusers.

One object of the invention is providing two or more perfumed formulations in a single diffuser that allows changing from one to the other as desired, without having to replace the container of the active substance, in a simple manner by simply turning the device.

Another object of the invention is providing in said diffuser of multiple volatile substances the possibility of regulating the global evaporator assembly of the diffuser, as well as allowing an individual regulation of each container comprising it.

Also the object of the invention is that the device has a simple construction, with a limited number of parts, without requiring the presence of electrical components, to provide an inexpensive construction as well as performing a clean, drip-free air freshening function.

### BACKGROUND OF THE INVENTION

A great diversity of perfume diffusers or air fresheners are currently known, both electrical and static, i.e. not electrical.

Within the class of static perfume diffusers may be cited U.S. Pat. Nos. 4,739,928, 4,621,768, 5,725,152 and 4,928,881.

The inventions described in the first two documents relate to air fresheners of the type including a wick in contact with the air freshening liquid, provided with means for regulating the evaporation of the liquid. The invention cited in first place, corresponding to U.S. Pat. No. 4,739,928, allows adjusting the emanation of the perfume by an emanator or absorbent pad attached to the neck of the container conforming the air freshener by means of a rotation of the lid covering the support in which said absorbent pad is housed; The air freshener described in U.S. Pat. No. 4,621,768 regulates the evaporation of the liquid by slits made in the lid, which also turns to vary the fragrance diffusing substance without altering the original shape of the device itself.

Another fragrance diffusion device with a wick is that described in the aforementioned U.S. Pat. No. 5,725,152, which can be used for a long time as the emanation means can be easily replaced, without deforming the air freshener, which is given a particular ornamental appearance with a flower-shaped lid. A flower bud shape is also used in the device of U.S. Pat. No. 4,928,881, which contains a scented liquid and a wick in which the wick and the liquid container may be separated from the base of the air freshener, which due to its peculiar shape can be used for other purposes, such as an ashtray or a flower pot.

As a final example of an non-electrical air freshener can be cited the International Patent Application PCT/ESO/3/00291, with the same holder as the present document, which claims an adjustable liquid air freshener having a cap incorporating means that prevent its disassembly from a stopper which, when turning without allowing its extraction from the device, opens the windows made in the cap to expose to the air a wick soaked in the liquid.

As regards known electrical diffuser devices for volatile substances with perfumes, it is also possible to cite the standards of the company applying for this patent on devices with heating or operating by a flow of perfumed air.

All of these fragrance diffusion devices and other air fresheners existing in the market have the drawback of containing a single chemical product emanating the perfume, with the disadvantage that in order to change the fragrance it is necessary to replace the product with the evaporable substances, even if it has not been exhausted, for another with a different scent. They therefore lack the possibility of including different fragrances in a single device without having to resort to purchasing another air freshening means (liquid, wick, pill, pad, etc.), replacing the one contained in the device.

### DESCRIPTION OF THE INVENTION

This invention solves the aforementioned drawbacks, among others, in a fully satisfactory manner in each and every aspect considered, providing advantages with respect to the antecedents.

Specifically, the invention relates to a diffuser of multiple active substances that comprises two or more containers, characterised by having orientations conducive to the release of the volatile substance and other orientations that are not conducive to its release, said containers located at relatively symmetrical positions with respect to a central body, together conforming the air freshener device, so that in a normal position one or several of the containers are in a position conducive to release of the volatile substance while the other(s) is(are) in a position not conducive to its release.

Depending on the total number of containers and the form of each one, the diffuser of multiple fragrances can adopt diverse configurations with a geometry approximately symmetrical with respect to the central body. Specifically, with two identical containers, each one with a hemispherical shape, joined symmetrically to the central body, the device of the invention can have a pleasant appearance as a spherical air freshener that allows its use in a household room as a discreet decorative element. However, the possible geometrical shapes (cubes, etc.) are endless, changing the appearance and number of containers of perfumed substances.

Each one of the multiple containers of the device holds volatile substances of a different nature that can produce different effects, not only as regards the beneficial effects of aromatherapy but also other advantages by introducing active principles of insecticides, bactericides, etc. in some of the containers.

The manner in which the fragrance emanated at a given moment by the diffuser from one of its various containers is also simple and quick to change, by simply changing the position of the device turning it about one of its axes of symmetry, so that the container emanating perfume stops doing so and the diffusion of substance is performed by another container or other containers included in the device of the invention. More specifically, when the diffuser comprises two containers, in the resting state only one of them emanating substance (the upper or lower one, according to the means of diffusion of active substances disposed internally), the change of aroma or emanation of active principles of insecticides or other products is simply performed by turning over the air freshener.

The invention disclosed can have several embodiments:

One alternative embodiment is manufacturing the diffuser of multiple fragrances with two containers or bottles with identical shape placed symmetrically about a central body, from where the volatile substances, perfume or other active substances contained in the bottles are evaporated. The mouth of each bottle is closed by a porous stopper body with physical properties such that this body can be soaked in the air freshener or insecticide liquid, etc. but does not let the substance out in its liquid form, so that only the vapour of the substance is released from the container. The configuration of the central body and the support part constituted by it between the two containers is such that it allows screwing the stopper of each bottle independently, so that the release of each substance contained in them can be regulated. At rest, the bottle that is upside down will diffuse its fragrance, while as the porous body of the one right side up is not in contact with the liquid this bottle will not diffuse the air freshening scent. In addition, the diffuser is provided with a global regulation means in its central body consisting of a regulation part or ring that opens and closes the central evaporation space.

Alternatively, the bottles are closed by previously sealed semi-permeable membranes that are activated immediately prior to their first use by removing a thin aluminium foil that preserves their properties intact while not in use.

Another alternative for closing said bottles is the use of stopper/wick assemblies with a wick design such that it can only be fed with air freshening liquid, insecticide, etc. in its final few millimetres, this is, near the bottom of the bottle, for example by the presence of a sheath around the wick except in this final area. In this way, when a bottle such as this is placed upside down when the diffuser is at rest the wick will not obtain liquid and thus no diffusion will take place, the bottle that is right side up with its wick immersed in the air freshening liquid producing the emanation of volatile substances.

In general, the two bottles contain different perfumed formulations, although they can also be identical. Another option is that one bottle contains the air freshener as such, while the other additionally includes an insecticide, bactericide, etc. to provide when desired the additional advantageous effect of the diffuser device formed by the two containers, without changing or affecting the scent it provides.

One improvement of the multiple fragrances device object of the invention is that its central body is provided with a moving element which, when turned around to leave the bottle that was previously above on the bottom and vice versa, this moving element closes the bottle (for example by gravity when the non-diffusing bottle to be closed is the bottom one, such as when using a porous stopper for the bottles, as in the first and preferred alternative described) which will no longer emanate any volatile substances, to ensure that the evaporation in the bottle without emanation is minimal, or ideally inexistent.

Additionally, the diffuser device of multiple fragrances may be static or electrical, incorporating a mechanism among known mechanisms (heat, air flow, vibration, etc.) or one specifically implemented to enhance the evaporation of the substances employed.

The device can be turned to change the relative position of its bottles in an automated manner, such as by pressing a button or by a timed program, by mounting it on a suitable support for this purpose. The diffuser of multiple fragrances can thus be adapted to a support that facilitates the manual rotation to reposition the bottles or to perform this automatically.

A last variant of this device object of the invention is its configuration with more containers in a geometry with a number of sufficiently large faces, so that the assembly of the containers and the central body by which they are joined, constituting the diffuser, may rest on any of its faces, allowing multiple different combinations in addition to the two pure fragrances provided in the basic configuration, for perfumes and products with other effects (against bacteria and insects, medicinal, etc.).

Even in its most basic variant, the invention has several advantages:
It allows changing the perfumed formulation emanated by the air freshener in a simple and fast manner, simply by turning the device manually or automatically using a suitable support whenever the user wishes, without having to replace the containers or the products included in them, simply changing its position by moving the diffuser assembly in an appropriate manner, usually by a rotation of 180°
The change of fragrance is achieved by a very simple technical solution, with a limited number of parts and without the need, although this is optional, of using any electrical components
The diffuser of multiple volatile substances offers the possibility of a global regulation of the evaporator assembly and an individual regulation of the evaporation of each active substance for each container comprising the assembly
The device is easily manufactured and operates cleanly, being provided with internal means that prevent dripping of the perfume liquid or insecticidal, bactericidal, medicinal, etc. formulae.

### DESCRIPTION OF THE DRAWINGS

To complete the description being made and in order to aid a better understanding of the characteristics of the invention, according to a preferred embodiment, a set of drawings is accompanied as an integral part of the description where, for purposes of illustration and in a non-limiting manner, the following is shown:

FIG. 1.—Shows a perspective view of an embodiment of the device of the invention formed by two identical containers joined symmetrically to form a sphere by a central body provided with a global regulation ring with windows made between the locking tabs that allow the rotation of one container with respect to the other, the assembly also free to rotate 180° about the vertical axis of symmetry to place on or the other container on the top or on the bottom, depending on which substance is to be diffused.

FIG. 2.—Shows an exploded view of one of the containers of the device object of the invention for the embodiment shown in the previous figure, revealing the specific closure and regulation means of the container.

FIG. 3.—Shows a perspective view of said embodiment of the device object of the invention, with the two containers separated to show the porous body that acts as a stopper in the container placed on the bottom.

FIG. 4.—Shows a longitudinal section of the two containers that form said embodiment of the device of the invention, without its central support body, showing the active liquid substance in the top container that is diffused in contact with the porous body, while the bottom container does not diffuse as its liquid substance is not in contact with the porous body.

FIG. 5.—Shows a longitudinal section of the two containers forming another embodiment of the device of the invention, without its central support body and having wicks instead of porous bodies, showing the wick in the bottom container that diffuses in contact with the liquid active substance, while the top container is not diffusing as the free end of its wick is not in contact with the liquid substance it contains.

### PREFERRED EMBODIMENT OF THE INVENTION

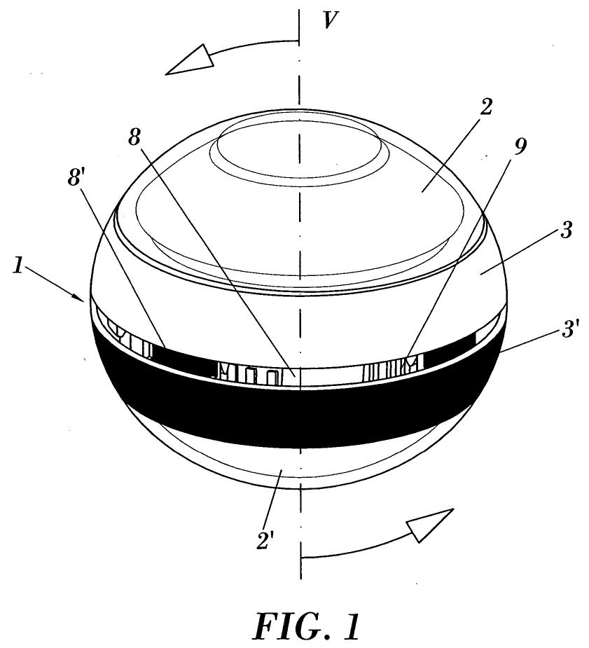

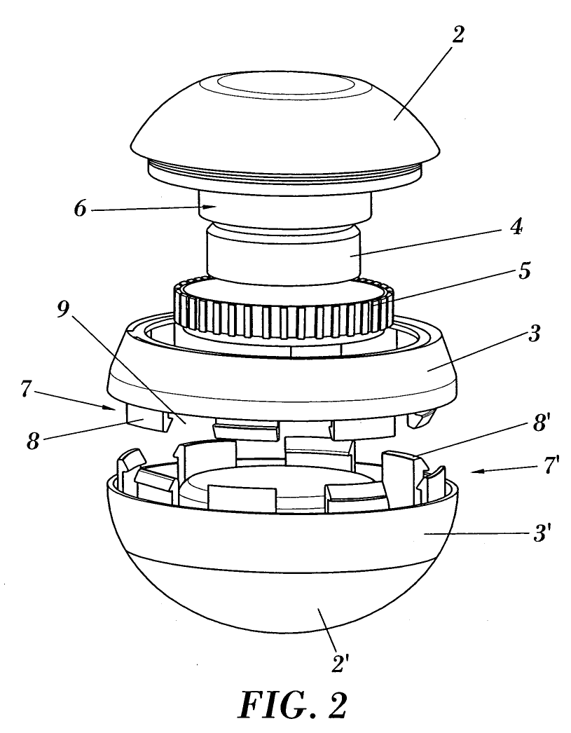

In view of FIGS. 1 and 2, it is possible to describe as one of the possible embodiments of the invention a diffuser of multiple volatile substances comprising two containers (2, 2′) placed in relatively symmetrical positions with respect to a central body (1) to which they are joined by the corresponding supports (3, 3′) that constitute said central body (1).

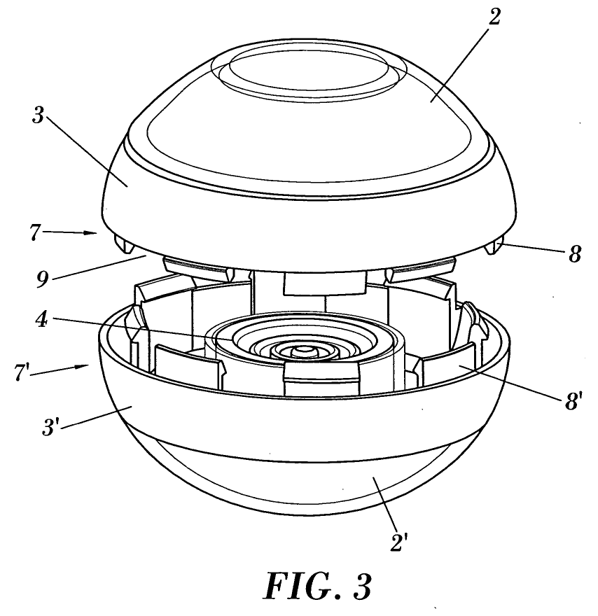

Each container (2, 2′) is closed by a diffuser body of the active substance (4), which preferably is a porous material with relief to increase the evaporation surface of the active volatile substances, as shown in FIG. 3, acting as a stopper and due to its porous nature preventing the container (2, 2′) from dripping when it is placed on top, placing the diffuser device at rest. To achieve the seal the active substance diffuser body (4) fits snugly in the mouth (6) of the container (2, 2′).

In addition, each container (2, 2′) has a cap screwed on its mouth (6) that acts as an individual means of regulation for the diffusion of the active volatile substance in each container (2, 2′).

In turn, the central body (1) acts as a global regulation means for the diffusion of volatile active substances, opening or closing the central evaporation opening. To do so, it is provided with an upper regulation ring (7) and a lower regulation ring (7′) respectively provided with locking tabs (8, 8′) orientated so that they lock in each to allow regulating the evaporation of the volatile active substances when the upper regulation ring (7) is turned about the lower regulation ring (7′), as the turning motion opens to a greater or lesser extent the windows (9) made in the corresponding locking tabs (8, 8′).

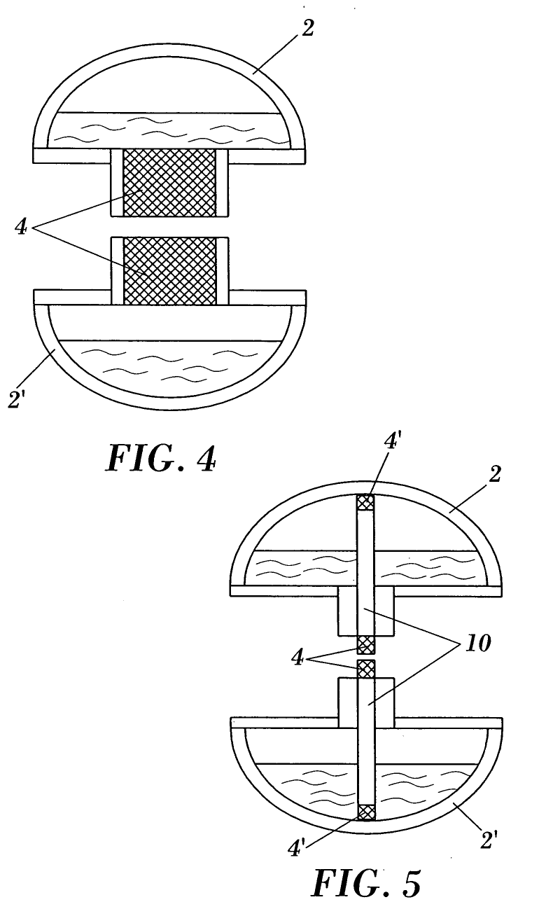

FIG. 4 shows the two containers (2, 2′) of this preferred embodiment in a longitudinal section, illustrating the form in which the diffusion takes place from the container (2) placed on top, as the liquid active substance comes in contact with the diffuser body (4), which in this case is a porous body that is impregnated with the liquid, without letting the liquid out yet allowing the exit of its vapour as the liquid active substance impregnating it evaporates. Meanwhile, the container on the bottom (2′) is not in a diffusion state as its diffuser body (4) is not in contact with the liquid. In short, only the container (2) on the top will diffuse.

Another embodiment of the invention is shown in FIG. 5, where the active substance diffuser body (4) is a wick with a free end (4′) that only comes in contact with the liquid in the container on the bottom (2′), which is where the diffusion takes place, while the container (2) on top cannot diffuse as the free end (4′) of its wick is not in contact with the liquid substance and the rest of the wick is protected by a sheath (10) that prevents the wicks from being impregnated except in the aforementioned free end (4′). Thus, in the embodiment having a wick as the diffusing body (4) the bottom container (2′) performs the diffusion.

In view of this text and the set of drawings, the invention has been described according to some preferred embodiments for it, although it will be obvious to an expert in the field that many variations can be made to these preferred embodiments within the object of the invention claimed.

The terms used in this memory should be understood in a wide and non-limiting sense.

1.- diffuser of multiple volatile substances such as an air freshener, insecticide, bactericide etc. characterised in that it comprises at least two containers (2, 2′) housing said volatile substances, joined in a relatively symmetrical disposition about a central body (1) where the evaporation of said substances occurs, said body consisting of corresponding supports (3, 3′) for the respective containers (2, 2′) so that when this device is in a resting position, at least one of the containers (2, 2′) diffuses volatile active substances by an active substance diffuser body (4) inserted in the mouth (6) through said central evaporation area by the global regulation means, while the remaining containers are not in a suitable position for diffusing the substances they contain, thereby providing a simple and fast manner of changing the fragrance, simply modifying the relative position of the containers (2, 2′) by turning the device.

2.- Diffuser of multiple volatile substances according to claim 1, characterised in that the active substance diffuser body (4) is a porous material that prevents the active substance from passing while allowing its vapour to pass.

3- Diffuser of multiple volatile substances according to claim 1, characterised in that the active substance diffuser body (4) is a semi-permeable membrane.

4.- Diffuser of multiple volatile substances according to claim 1, characterised in that the active substance diffuser body (4) is a wick with a sheath (10) that can be impregnated with the active substance of the container (2, 2′) only near the base of the container at a free end (4′).

5.- Diffuser of multiple volatile substances according to any of the previous claims, characterised in that said central body (1) also constitutes a global regulation means for the diffusion of the volatile active substances that opens or closes the central evaporation area.

6.- Diffuser of multiple volatile substances according to claim 5, characterised in that said global regulation means comprises an upper regulation ring (7) and a lower regulation ring (7′) provided with corresponding locking tabs (8, 8′), correspondingly orientated to lock in each other allowing to turn the upper regulation ring (7) about the lower regulation ring (7′) to open or close windows (9) defined between the corresponding locking tabs (8, 8′).

7.- Diffuser of multiple volatile substances according to any of the previous claims, characterised in that the containers (2, 2′) incorporate individual regulation means for the diffusion of the active volatile substances.

8.- Diffuser of multiple volatile substances according to claim 7, characterised in that said individual regulation means for the diffusion of the active volatile substances in each of the containers (2, 2′) consist of a cap (5) screwed on the mouth (6) of the container (2, 2′).

9.- Diffuser of multiple volatile substances according to any of the previous claims, characterised in that it incorporates an electrical device having means to enhance the evaporation of the volatile substances in the central area by means of heat.

10.- Diffuser of multiple volatile substances according to any of the previous claims, characterised in that it incorporates an electrical device having means to enhance the evaporation of the volatile substances in the central area by generating an air flow.

11.- Diffuser of multiple volatile substances according to any of the previous claims, characterised in that it incorporates an electrical device having means to enhance the evaporation of the volatile substances in the central area by means of vibration.

12.- Diffuser of multiple volatile substances according to any of the previous claims, characterised in that the containers/central body assembly described is mounted on a motorised structure that allows changing the positions of the containers in an automated or programmed manner.
